(19) United States
(12) Patent Application Publication
(10) Pub. No.: US 2024/0189205 A1
(43) Pub. Date: Jun. 13, 2024

(54) ENRICHED TOPICAL COSMETIC COMPOSITION

(71) Applicant: TruSkin Partners, Inc., Seattle, WA (US)

(72) Inventor: Jalshree Trivedi, Fremont, CA (US)

(73) Assignee: TruSkin Partners, Inc., Seattle, WA (US)

(21) Appl. No.: 18/535,023

(22) Filed: Dec. 11, 2023

Related U.S. Application Data
(60) Provisional application No. 63/386,806, filed on Dec. 9, 2022.

(51) Int. Cl. A61K 8/67; A61K 8/19; A61K 8/34; A61K 8/35; A61K 8/36; A61K 8/37; A61K 8/49; A61K 8/58; A61K 8/73; A61K 8/9794; A61Q 19/00; A61Q 19/08

(52) U.S. Cl. CPC A61K 8/67; A61K 8/19; A61K 8/345; A61K 8/355; A61K 8/361; A61K 8/37; A61K 8/494; A61K 8/4973; A61K 8/58; A61K 8/673; A61K 8/675; A61K 8/676; A61K 8/678; A61K 8/735; A61K 8/9794; A61Q 19/007; A61Q 19/08; A61K 2800/48

(57) ABSTRACT

Provided herein are cosmetic composition for skincare, comprising a unique blend of plant-derived vitamins, minerals and electrolytes to reduce prevent, reduce and treat damage to the skin.

### CROSS REFERENCE TO RELATED APPLICATIONS

This application claims the benefit of U.S. Provisional Application No. 63/386,806 entitled “ENRICHED TOPICAL COSMETIC COMPOSITION” and filed on Dec. 9, 2022, the contents of which are hereby incorporated by reference in their entirety.

### BACKGROUND

### 1. Field

The present invention relates to topical cosmetic compositions for skincare.

### 2. Background

Skin is the bodies first line of defense against a range of environmental assaults including oxidative stress, ultraviolet (UV) rays, high energy visible light (violet-blue) emitted by devices such as TV/computer/smartphone screens, as well as harmful chemical agents found in airborne pollution such as smog and cigarette smoke. Additionally, skin is also subject to structural damage due to aging and other biological factors. It is therefore desirable to adequately nourish and hydrate the skin using superior ingredients.

There is a continuing desire to provide compositions and methods of treatment that can improve the appearance of skin, rejuvenate skin after damage from environmental and oxidative stresses, and reduce the appearance of wrinkles and other age related symptoms on skin, while causing minimal skin irritation and side effects. These and other improvements over the art are provided by the present invention.

### SUMMARY OF THE INVENTION

In some aspects, the current disclosure encompasses a topical composition, comprising: at least four vitamins, at least one electrolyte, at least one mineral, and at least one excipient. In some aspects, the topical composition comprises at least five, at least six, at least seven or more vitamins. In some aspects, the vitamin is plant derived. In some aspects, the vitamins are selected from a group consisting of: linoleic acid and linolenic acid fatty acids (vitamin F), niacinamide (vitamin B3), sodium ascorbyl phosphate (vitamin C), cholecalciferol (vitamin D3), phytomenadione (vitamin K1), calcium D-pantothenic acid (vitamin B5), tocopheryl acetate (Vitamin E Acetate), tocopherol (Vitamin E), or a suitable derivative or variant thereof. In some exemplary aspects the topical composition comprises vitamins at the following range of weight percentage of the topical composition: linoleic acid and linolenic acid fatty acids (vitamin F) about 0.01-to about 2%; niacinamide (vitamin B3)—about 0.01% to about 5%; sodium ascorbyl phosphate (vitamin C)—about 0.01% to about 20%; cholecalciferol (vitamin D3)—about 0.01% to about 2%; phytomenadione (vitamin K1) about 0.01% to about 2%; D-Calcium pantothenic acid (vitamin B5) about 0.01% to about 5%; and tocopheryl acetate (Vitamin E Acetate) about 0.01% to about 5%.

In some aspects, the topical composition comprises at least one electrolyte comprises a magnesium salt, a potassium salt, a calcium salt, or any combination thereof. In some aspects, the at least one electrolyte comprises magnesium chloride, sodium chloride, potassium chloride, calcium chloride, or any combination thereof and the at least one electrolyte is present in an amount of about 0.01 to about 2%. In some aspects, the least one mineral is zinc pyrrolidone carboxylic acid (PCA) present in an amount of about 0.01 to about 4%.

In some aspects, the disclosed compositions also comprise at least one excipients comprising any one or more of humectants, emollients, solvents, oils, fats, thickening agents, buffers, preservatives, coloring agents, fragrance or any combination thereof. In some aspects the excipients comprise water, glycerin, glyceryl stearate, Aloe barbadensis leaf juice, sorbitan oliviate, hexylene glycol, jojoba esters, magnesium stearate, corn oil, capryly glycol, allantoin, phenoxyethanol, caprylyl glycol, ethylhexylglycerin, or any combination thereof. In some aspects, the excipients are present in the range of water about 80 wt % to about 90 wt % of water, about 1.5 wt % to about 2 wt % of glycerine, about 1.5 wt % to about 2.5 wt % of glyceryl stearate, about 1 wt % to about 3 wt % of Aloe barbadensis leaf juice, about 0.5 wt % to about 1.5 wt % of sorbitan oliviate, less that about 1 wt % of hexylene glycol, less that about 1 wt % jojoba esters, less that about 1 wt % magnesium stearate, less that about 1 wt % of corn oil, less that about 1 wt % allantoin, less that about 1 wt % capryly glycol, less that about 1 wt % phenoxyethanol, less that about 1 wt % ethylhexylglycerin.

In some aspect, the topical composition comprises exemplary formulation 1.

In some aspects, the topical composition is applied to the surface on the subject to prevent, reduce or treat one or more of fine lines, wrinkles, moisture loss, hyperpigmentation, uneven skin tone, crepe skin, collagen loss, oxidation, uneven skin tone, skin redness, or free radical formation. In some aspects, the topical composition is administered to the surface on the subject to enhance one or more of hydration, passage for absorption, skin brightness, or healthy glow. In some aspects, the composition is in the form of a solids, liquids, suspensions, semisolids (such as creams, gels, pastes or “sticks”), powders or finely dispersed liquids such as sprays or mists.

In some aspects, the current disclosure also encompasses a method of improving the condition of the skin of a subject in need thereof comprising, applying to the subject a topical composition disclosed herein. In some aspects, the subject is a human. In some aspects, the current disclosure also encompasses a kit for skincare comprising the topical compositions as provided here and instructions for use.

### DETAILED DESCRIPTION

The following detailed description references the accompanying drawings that illustrate various aspects of the present inventive concept. The drawings and description are intended to describe aspects and aspects of the present inventive concept in sufficient detail to enable those skilled in the art to practice the present inventive concept. Other components can be utilized, and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based at least in part on the unexpected result that a particular blend of eleven ingredients comprising vitamins, minerals, electrolytes, and certain excipients is sufficient to replenish the skin with the nutrients that it loses naturally over time. The composition as disclosed herein comprises a blend of vitamins B₂, B₅, C, D, E, F, and K to promote overall cell health while improving texture and tone, zinc to support collagen repair and help strengthen skin's barrier, and magnesium, potassium, and calcium to help skin retain moisture and regulate pH for gentle, skin-friendly formulas. In some aspects, other ingredients can be added for desired properties, for example suitable consistency, color and desired applicability profile.

### I. Compositions

In some aspects, the current disclosure encompasses a topical composition comprising at least four, at least five, at least six, at least seven or more vitamins. Non-limiting examples of vitamins that can be used in the compositions provided herein include B₂, B₅, C, D, E, F, and K. More specifically, the compositions may comprise linoleic acid and linolenic acid fatty acids (vitamin F), niacinamide (vitamin B3), sodium ascorbyl phosphate (vitamin C), cholecalciferol (vitamin D3), phytomenadione (vitamin K₁), Calcium D-pantothenic acid (vitamin B₅), tocopheryl acetate (Vitamin E Acetate), tocopherol (Vitamin E), or a suitable derivative or variant thereof. In some aspects, the vitamins used in the current disclosure are derived from plants.

In some aspects, the topical composition may comprise vitamin F. Vitamin F is comprised of two types of essential fatty acids—alpha-linolenic acid (ALA) and linoleic acid (LA). These are omega-3 and omega-6 fatty acids which can be found in many plant-based food sources such as oils, nuts and seeds. In some aspects, vitamin F can be sourced from any suitable source including plants, animals, microorganisms, or synthetic sources. In some aspects of the current disclosure, the vitamin F can be sourced from one or more plants. In some aspects, the fatty acids of vitamin F can be derived from nuts; non-limiting examples of which include walnuts, hazelnuts, cashews, almonds, pine nuts, seed; non-limiting examples of which include flaxseed, chia seeds, sunflower seeds, plant derived oils; non-limiting examples of which include flaxseed oil, canola oil, walnut oil, and soybean oil. In some aspects, the inclusion of vitamin F to the composition provides multiple benefits as provided in the current disclosure. In some aspects, vitamin F may help in preserving skin's barrier function against UV damage, air pollutants such as smoke that increase free radicals that result in wrinkles and aging. In some aspects, vitamin F helps retain moisture in the skin.

In some aspects, the topical composition comprises vitamin F in the amount of about 0.01%-2% by weight. In some aspects, the topical composition comprises vitamin F in the amount of about 0.01% to about 0.05%, or about 0.05% to about 0.1%, or about 0.1% to about 0.5%, or about 0.5% to about 1%, or about 1% to about 1.5%, or about 1.5% to about 2% by weight. In some aspects, the topical composition comprises about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% of vitamin F by weight. In some aspects, vitamin K is less than 1% of the formulation.

In some aspects, the topical composition may comprise vitamin B₃. Vitamin B₃ includes three forms or vitamers: niacin (nicotinic acid), nicotinamide (niacinamide), and nicotinamide riboside. In some aspects, the topical composition comprises niacinamide. In some aspects, niacinamide can be sourced from any suitable source including plants, animals, microorganisms or synthetic sources. In some aspects, the niacinamide is sourced from plants, for example from nutritional yeast, seaweed, hemp, chia, wheat, buckwheat, sesame, pine nuts, rye grain, grains, avocado, almonds, flaxseed, nuts, cereals, quinoa, yeast extract, wild rice, corn, brown rice, lentils, vegetables like squash, potatoes, asparagus, bell peppers, carrots, and fruits. In some aspects, the inclusion of vitamin B₃ to the composition provides several benefits as disclosed herein. These include, but are not restricted to building proteins and ceramides, soothing the skin, improving texture and hydration by preventing moisture loss, improving skin elasticity, and minimizing pore appearance. In addition, niacinamide (vitamin B3) may help improve the appearance of aging facial skin, reduce photodamage and lower the sebum which helps in minimizing the pore size.

In some aspects, the topical composition comprises vitamin B3 in the amount of about 0.01%-5% by weight. In some aspects, the topical composition comprises vitamin B3 in the amount of about 0.01% to about 0.05%, or about 0.05% to about 0.1%, or about 0.1% to about 0.5%, or about 0.5% to about 1%, or about 1% to about 1.5%, or about 1.5% to about 2%, or about 2% to about 2.5%, or about 2.5% to about 3%, or about 3% to about 3.5%, or about 3.5% to about 4%, or about 4% to about 4.5%, or about 4.5% to about 5% by weight. In some aspects, the topical composition comprises about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5% of vitamin B₃ by weight. In some aspects, vitamin B₃ is less than 1 wt % of the formulation.

In some aspects, the topical composition disclosed herein may comprise vitamin B₅ (pantothenic Acid). Pantothenic Acid is a water-soluble B vitamin. In some aspects, the vitamin B₅ can be obtained from any suitable source including plants, animals, microorganisms or synthetic sources. In some aspects, the pantothenic acid is sourced from synthetic sources. In some aspects, the pantothenic acid is sourced from plant-based sources including fruits, vegetables, nuts or cereals. In some aspects, the inclusion of vitamin B₅ to the composition provides multiple benefits as provided herein. In some aspects, vitamin B₅ may help in promoting overall healthy skin and increasing skin hydration by reducing trans epidermal water loss. It also functions as a humectant.

In some aspects, the topical composition comprises vitamin B₅ in the amount of about 0.01%-5% by weight. In some aspects, the topical composition comprises vitamin B₅ in the amount of about 0.01% to about 0.05%, or about 0.05% to about 0.1%, or about 0.1% to about 0.5%, or about 0.5% to about 1%, or about 1% to about 1.5%, or about 1.5% to about 2%, or about 2% to about 2.5%, or about 2.5% to about 3%, or about 3% to about 3.5%, or about 3.5% to about 4%, or about 4% to about 4.5%, or about 4.5% to about 5% by weight. In some aspects, the topical composition comprises about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9% or 5% of vitamin B₅ by weight. In some aspects, vitamin B₅ is less than 1 wt % of the formulation.

In some aspects, the topical composition may comprise vitamin D (cholecalciferol). Vitamin D is a group of fat-soluble secosteroids. The most important compounds in this group are vitamin D₃ (cholecalciferol) and vitamin D₂ (ergocalciferol). In some aspects, the topical composition comprises cholecalciferol. In some aspects, cholecalciferol can be sourced from any suitable source including plants, animals, microorganisms, or synthetic sources. In some aspects, the cholecalciferol is sourced from vegetarian sources for example milk or mushrooms. In some aspects, vitamin D helps reduce acne, and prevent, reduce or treat eczema and psoriasis.

In some aspects, the topical composition comprises cholecalciferol in the amount of about 0.01% to about 0.05%, or about 0.05% to about 0.1%, or about 0.1% to about 0.5%, or about 0.5% to about 1%, or about 1% to about 1.5%, or about 1.5% to about 2%. In some aspects, the topical composition comprises about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% of cholecalciferol by weight. In some aspects, the cholecalciferol is less than 1 wt % of the formulation.

In some aspects, the topical composition may comprise vitamin C. In some aspects, ascorbate salts such as sodium ascorbate and calcium ascorbate are good sources of vitamin C. In some aspects, the vitamin C compound as used in the current disclosure may be sodium ascorbyl phosphate. Vitamin C is a water-soluble vitamin. In some aspects, the vitamin C can be sourced from plants, animals, microorganisms, or synthetic sources. In some aspects, the vitamin C is sourced from plants. In some aspects, vitamin C can be sourced from fruits and vegetables non-limiting examples of which include citrus fruits (oranges, kiwi, lemon, grapefruit), peppers, strawberries, tomatoes, cruciferous vegetables, and potatoes. In some aspects, vitamin C provides several essential properties to the composition as provided herein. Vitamin C is an antioxidant, and is required for collagen synthesis, which occurs in the dermis (lowest) layer of the skin. Sodium ascorbyl phosphate (Vitamin C) used in the compositions herein is the most stable version for the topical skincare and works well in the skin balanced PH system. Vitamin C can help prevent acne, neutralize free radicals, and has anti-inflammatory properties. It also helps improve skin tone, brighten the skin, and reduce hyperpigmentation.

In some aspects, the topical composition may comprise vitamin C in the amount of about 0.01%-20% by weight. In some aspects, the topical composition comprises vitamin C in the amount of about 0.01% to about 0.05%, or about 0.05% to about 0.1%, or about 0.1% to about 0.5%, or about 0.5% to about 1%, or about 1% to about 1.5%, or about 1.5% to about 2%, or about 2% to about 2.5%, or about 2.5% to about 3%, or about 3% to about 3.5%, or about 3.5% to about 4%, or about 4% to about 4.5%, or about 4.5% to about 5%, or about 5% to about 10%, or about 10% to about 15%, or about 15% to about 20%. In some aspects, the topical composition comprises about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 6%, 7%, 8%, 9%, 10%, 11%, 12%, 13%, 14%, 15%, 16%, 17%, 18%, 19%, or 20% of vitamin C by weight. In some aspects, vitamin C is less than 1 wt % or less than 5% of the formulation.

In some aspects, the topical composition may comprise vitamin E (tocopherol). Vitamin E is a fat-soluble vitamin with several forms, but alpha tocopherol is the only one used by the human body. Its main role is to act as an antioxidant, scavenging loose electrons—so-called “free radicals”—that can damage cells. Vitamin E is essential for maintaining the healthy skin. As an antioxidant vitamin E primarily reacts with the oxygen reactive species. This plays an important role in photo protection by preventing the damage of free radicles in the environment. The lower the vitamin E content in the skin, higher is the risk of UV damage. As we age the vitamin E concentration in stratum corneum also declines. As such, addition of vitamin E to the disclosed composition can be beneficial. In some aspects, the vitamin E can be sourced from plants, microorganisms, or synthetic sources. In some aspects, the vitamin E is sourced from oils, nuts, seeds, fruits, and vegetables, for example wheat germ, sunflower, safflower, soybean oil, almonds, peanuts, greens, pumpkin, peppers, mango, and avocado.

In some aspects, the topical composition comprises vitamin E in the amount of about 0.01%-2% by weight. In some aspects, the topical composition comprises vitamin E in the amount of about 0.01% to about 0.05%, or about 0.05% to about 0.1%, or about 0.1% to about 0.5%, or about 0.5% to about 1%, or about 1% to about 1.5%, or about 1.5% to about 2% by weight. In some aspects, the topical composition comprises about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% of vitamin E by weight. In some aspects, vitamin E is less than 1 wt % of the formulation.

In some aspects, the topical composition as disclosed herein may comprise vitamin K. refers to structurally similar, fat-soluble vitamers. Chemically, the vitamin K family comprises 2-methyl-1,4-naphthoquinone (3-) derivatives. Vitamin K includes two natural vitamers: vitamin K₁ (phylloquinone) and vitamin K₂ (menaquinone). Vitamin K₂, in turn, consists of a number of related chemical subtypes, with differing lengths of carbon side chains made of isoprenoid groups of atoms. In some aspects, the compositions of the current disclosure comprise a commonly used form of vitamin K, phytonadoine. Phytonadione used in the current disclosure may be sourced from plants, for example green leafy vegetables, olive oil, or soybean oil. Vitamin K helps the collagen in the body. Healthy collagen production leads to smooth, plumped, healthy and younger looking skin. Vitamin K is high on antioxidant and is involved in tissue renewal and cell growth control. This significantly leads to softer and healthy skin. Vitamin K helps the body to produce the new skin cells which replaces old skin cells on a cyclic basis. Because of the ability to maintain the collagen in the skin, vitamin K is very effective for fine lines and wrinkles as decrease of collagen and elastin in the skin is a root cause of aging effects. Vitamin K also helps in wound healing.

In some aspects, the topical composition comprises phytonadione or equivalent source of vitamin K in the amount of about 0.01%-2% by weight. In some aspects, the topical composition comprises phytonadione or vitamin K in the amount of about 0.01% to about 0.05%, or about 0.05% to about 0.1%, or about 0.1% to about 0.5%, or about 0.5% to about 1%, or about 1% to about 1.5%, or about 1.5% to about 2% by weight. In some aspects, the topical composition comprises about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% of phytonadione or vitamin K by weight. In some aspects, phytonadione or vitamin K is less than 1 wt % of the formulation.

In some aspects, the topical compositions disclosed herein further comprise at least one mineral. In some aspects, the at least one mineral is zinc. In some aspects, zinc is added in the form of zinc pyrrolidone carboxylic acid (PCA). Zinc PCA can be added to the composition and provides several beneficial properties including skin tightening, reduced sun damage, enhanced protection against environmental hazards, and anti-inflammatory properties that reduce breakouts and swelling.

In some aspects, the topical compositions disclosed herein may comprise Zinc PCA in amounts of about 0.01% to about 4% by weight. In some aspects, the topical composition comprises Zinc PCA in the amount of about 0.01% to about 0.05%, or about 0.05% to about 0.1%, or about 0.1% to about 0.5%, or about 0.5% to about 1%, or about 1% to about 1.5%, or about 1.5% to about 2%, or about 2% to about 2.5%, or about 2.5% to about 3%, or about 3% to about 3.5%, or about 3.5% to about 4%, or about 4% by weight. In some aspects, the topical composition comprises about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, or 4% of Zinc PCA by weight. In some aspects, Zinc PCA is less than 1 wt % of the formulation.

In some aspects, the topical disclosed herein further comprise at least one electrolyte. Non-limiting examples of electrolytes include magnesium chloride, potassium chloride, sodium chloride, and calcium chloride. In some aspects, the topical composition disclosed herein comprises magnesium chloride, potassium chloride, sodium chloride, calcium chloride, or any combination thereof. In some aspects, electrolytes help the skin retain water and regulate pH. In some aspects, magnesium chloride is a safe food-grade ingredient for skin care applications and helps in preventing, reducing or treating atopic dermatitis, dryness, and roughness. In some aspects, potassium chloride is also useful in cleansing of skin of dirt and other substances. In some aspects, apart from being an electrolyte, sodium chloride is also added to the formulation as a binding agent. In some aspects, calcium chloride is also added as a viscosity increasing agent and as a cosmetic astringent.

In some aspects, the topical agent disclosed herein comprises the at least one electrolyte in the amount of about 0.01 wt % to about 2 wt %. In some aspects, the topical composition comprises at least one electrolyte in the amount of about 0.01% to about 0.05%, or about 0.05% to about 0.1%, or about 0.1% to about 0.5%, or about 0.5% to about 1%, or about 1% to about 1.5%, or about 1.5% to about 2% by weight. In some aspects, the topical composition comprises about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% of at least one electrolyte by weight. In some aspects, the at least one electrolyte is less than 1 wt % of the formulation.

In some aspects, the topical compositions disclosed herein further comprise at least one excipient. In some aspects, the at least one excipient may comprise any one or more of humectants, emollients, solvents, oils, fats, thickening agents, buffers, preservatives, coloring agents, fragrance, or any combination thereof. In some aspects, the at least one excipient may comprise Aloe barbadensis leaf oil, caprylyl glycol, ethylhexylglycerin glycerin, urea, hyaluronic acid, salicylic acid, alpha hydroxy acids (AHAs) such as glycolic acid and lactic acid, propylene glycol, honey, sorbitol, beeswax, olive oil, coconut oil, lanolin oil (isolanolin), petrolatum (petroleum jelly), mineral oil, glycerin, methyl glyceth, zinc oxide, goat milk, butyl stearate, diglycol laurate, C12-15 alkyl benzoate, Aloe vera palmitate, butylene glycol, ethylhexyl palmitate, isododecane, isohexadecane, isopropyl myristate, octyldodecanol, PEG-40 hydrogenated castor oil, pentylene glycol BIO, (1, 3) propanediol, allantoin, lecithin, caprylic capric triglyceride, octyl dodecanol, cetearyl alcohol, squalene, squalene, tocopheryl acetate, polyglyceryl-6 laurate, ceteareth-20, dimethicone, PEG-100 stearate, octyldodecanol, caprylyl glycol, dicaprylyl carbonate, myristyl myristate, hectorite gel, stearyl palmitate, polyamide 3, sorbitol, polyhydroxystearic acid, lauryl laurate, cyclopentasiloxane (silicone gel), glyceryl stearate, sorbitol olivate, Jojoba esters, polysorbate 60, laureth-4, potassium cetyl sulfate, cetyl alcohol, cetearyl alcohol, stearyl alcohol, glyceryl stearate, polyglyceryl-6 laurate, ceteareth-20, sodium lauryl lactylate, myristyl myristate, carbomer, polysorbate 80, pola wax, sorbitan stearate, gum arabic, brassica alcohol, carbomer 980 QD, sodium stearate, polyhydroxystearic acid, PEG-150 distearate, glyceryl oleate, emulsifying wax, glyceryl monooleate, phospholipids, polyoxyethylene alkyl ethers, polyoxyethylene, castor oil derivatives, sorbitan fatty acid esters, polyoxyethylene stearates, polyoxylglycerides, polysorbate, sorbitan esters, sorbitan olivate, hexylene glycol, carbopol 980, cetyl alcohol, stearic acid, carnauba wax, hydroxyethyl cellulose, guar gum, xanthan gum, gelatin, magnesium aluminum silicate, silica, bentonite, cetyl palmitate, ammonium acryloyldimethyltaurate, cetearyl alcohol, glucose-D, hectorite gel, stearyl palmitate, gum arabic, hydroxypropyl starch phosphate, tapioca starch, acrylates octylacrylamide copolymer, carbomer 940, polyamide 3, castor wax, hydroxypropyl methylcellulose, Caesalpinia spinosa gum, brassica alcohol, sodium stearate, polyhydroxystearic acid, tribehenin, arrowroot starch, rice starch, candelilla wax, beeswax, ozokerite wax, sunflower wax, PEG-150 distearate, polyacrylate crosspolymer-6, acrylates C10-30 alkyl acrylate cross polymer, hydroxypropyl guar, cyclopentasiloxane and (silicone gel), glycerin, propanediol, butylene glycol, ethoxydiglycol, isododecane, isohexadecane, octyldodecanol, 1,2-hexane diol, dicaprylyl carbonate, or any combination thereof.

In some aspects, the excipients comprise water, glycerin, glyceryl stearate, Aloe barbadensis leaf juice, sorbitan oliviate, hexylene glycol, jojoba esters, magnesium stearate, corn oil, capryly glycol, allantoin, phenoxyethanol, caprylyl glycol, ethylhexylglycerin, or any combination thereof. In some aspects, the topical composition comprises about 80 wt % to about 82 wt %, or about 82 wt % to about 84 wt %, or about 84 wt % to about 86 wt %, or about 86 wt % to about 88 wt %, or about 88 wt % to about 90 wt % of water. In some aspects, the composition comprises about 80%, about 81%, about 82%, about 83%, about 84%, about 85%, about 86%, about 87%, about 88%, about 89%, or about 90% of water by weight. In some aspects, the topical composition comprises about 1.5 wt % to about 2.5 wt % of glycerin. In some aspects, the topical composition comprises about 1.5 wt %, or about 1.6 wt %, or about 1.7 wt %, or about 1.8 wt %, or about 1.9 wt %, or about 2.0 wt %, or about 2.1 wt %, or about 2.2 wt %, or about 2.2 wt %, or about 2.3 wt %, or about 2.4 wt %, or about 2.5 wt % of glycerin. In some aspects, the topical composition comprises about 1.5 wt % to about 2.5 wt % of glyceryl stearate. In some aspects, the topical composition comprises about 1.5 wt % or about 1.6 wt %, or about 1.7 wt %, or about 1.8 wt %, or about 1.9 wt %, or about 2.0 wt %, or about 2.1 wt %, or about 2.2 wt %, or about 2.2 wt %, or about 2.3 wt %, or about 2.4 wt %, or about 2.5 wt % of glyceryl stearate. In some aspects, the composition comprises about 1 wt % to about 3 wt % or Aloe barbadensis leaf juice. In some aspects, the topical composition comprises about 1 wt %, or about 1.1 wt %, or about 1.2 wt %, or about 1.3 wt %, or about 1.4 wt %, or about 1.5 wt %, or about 1.6 wt %, or about 1.7 wt %, or about 1.8 wt %, or about 1.9 wt %, or about 2.0 wt %, or about 2.1 wt %, or about 2.2 wt %, or about 2.2 wt %, or about 2.3 wt %, or about 2.4 wt %, or about 2.5 wt %, or about 2.6 wt %, or about 2.7 wt %, or about 2.8 wt %, or about 2.9 wt %, or about 3.0 wt % of Aloe barbadensis leaf juice. In some aspects, the composition comprises less than 1 wt %, or less than 0.8 wt %, or less than 0.6 wt %, or less than 0.5 wt %, or less than 0.4 wt %, or less than 0.3 wt %, or less than 0.2 wt %, or less than 0.1 wt % of any one or more of sorbitan oliviate, hexylene glycol, jojoba esters, magnesium stearate, corn oil, capryly glycol, allantoin, Phenoxyethanol, caprylyl glycol, or ethylhexylglycerin.

In some aspects, the compositions disclosed herein may be in any suitable form including but not limited to solids, liquids, suspensions, semisolids (such as creams, gels, pastes or “sticks”), powders, or finely dispersed liquids such as sprays or mists. In some aspects, the compositions may packaged into bottles, tubes, tablets, applicators, or capsules.

### II. Methods of Using

In some aspects, the current disclosure also encompasses methods of using the compositions disclosed herein for improving the condition of the skin in a subject. In some aspects, the compositions disclosed herein can be used in a subject to prevent, reduce or treat fine lines, wrinkles, moisture loss, hyperpigmentation, uneven skin tone, crepe skin, collagen loss, oxidation, uneven skin tone, skin redness, and free radical formation.

In some aspects, the composition may be applied to the skin one or more times daily, one or more times weekly, one or more time monthly or at any interval for any duration of time. For example, as a liquid composition, 3-5 drops of the composition may be used daily or multiple times in a day. In some aspects, such use may provide reduction in visible fine lines and wrinkles by about 1 to 20% as estimated by methods known in the art. In some aspects, use of 1, or 2, or 3, or 4, or 5, or 6, or 7, or 8, or 9, or 10 drops daily can reduce one or more of fine lines, wrinkles, moisture loss, hyperpigmentation, uneven skin tone, crepe skin, collagen loss, oxidation, uneven skin tone, skin redness, and free radical formation. In some aspects, the composition can be applied to any external or exposed part of the body. For example, the composition can be applied to the face, hands, elbow, knees, legs, toes, neck, or fingers.

In some aspects, the subject is a human. In some aspects, the subject is a human of any age from 0-120 yrs.

### III. Kits

In some aspects, the current disclosure also encompasses kits comprising the topical composition disclosed herein and instructions for use.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to aspects of many different forms, it is intended that the present disclosure be considered as an example of the principles of the present inventive concept and not intended to limit the present inventive concept to the specific aspects shown and described. Any one of the features of the present inventive concept may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present inventive concept may include a variety of combinations and/or integrations of the aspects described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The term “topical composition” or “topical formulation” or “topical cosmetics” or “topical products” as used herein encompasses substances that may be applied topically to the skin or mucous membranes of humans or animals in order to alter the subject's appearance, to protect the subject from the environment, or to produce a biological change in the skin or other tissue for therapeutic, preventive or cosmetic purposes. Topical products occur in a variety of forms, including solids, liquids, suspensions, semisolids (such as creams, gels, pastes, or “sticks”), powders, or finely dispersed liquids such as sprays or mists. Examples of topical products commonly classified as “cosmetics” include skin care products such as creams, lotions, moisturizers, serums, and “treatment cosmetics” such as exfoliants and/or skin cell renewal agents.

The term “emollient” as used herein encompasses ingredients in skin creams, lotions, moisturizers or ointments that form a film on the skin. These ingredients can relieve dryness, itching, and scaling. Emollients can help the skin feel more comfortable if you have eczema, psoriasis, dry, or sensitive skin.

The term “humectant” as used herein includes a substance that draws water into the skin, hair, or nails. In the skin, this may come from the deeper layers, or from the air if it is humid enough. Humectants are useful for adding hydration without feeling heavy or oily. Humectants include ingredients such as glycerin, urea, AHAs, and hyaluronic acid.

### Examples

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

### Exemplary Formulation 1 by Wt %

Water (Aqua): 82-90%
Glycerin: 3-6%
Glyceryl Stearate: 1.5-2.5%
Aloe barbadensis Leaf Juice: 1-3%
Sorbitan Olivate: 0.5-1.5%
Hexylene Glycol: <1%
Linoleic Acid: 0.01-2%;
Linolenic Acid: 0.01-2%;
Niacinamide: 0.01%-5%;
Sodium Ascorbyl Phosphate: <1%;
Allantoin: <1%;
Sodium Hyaluronate: <1%;
Cholecalciferol: 0.01%-2%;
Jojoba Esters: <1%;
Magnesium Stearate: <1%;
Phytonadione: 0.01%-2%;
Zea mays (Corn) Oil: <1%;
D-Calcium Pantothenate: 0.01%-5%;
Zinc PCA: <1%;
Carbomer: <1%;
Magnesium Chloride: <1%;
Potassium Chloride: <1%;
Sodium Chloride: <1%;
Calcium Chloride: <1%;
Phenoxyethanol: <1%;
Tocopheryl Acetate: 0.01%-5%;
Caprylyl Glycol: <1%;
Ethylhexylglycerin: <1%; and
Tocopherol: <1%.

### Exemplary Formulation II by Wt %

Linoleic acid and linolenic acid fatty acids (vitamin F): 0.01-2%;
Niacinamide (vitamin B3): 0.01%-5%;
Sodium ascorbyl phosphate (vitamin C): 0.01%-20%;
Cholecalciferol (vitamin D3): 0.01%-2%;
Phytomenadione (vitamin K₁): 0.01%-2%;
D-Calcium pantothenic acid (vitamin B₅): 0.01%-5%;
Tocopheryl acetate (Vitamin E Acetate): 0.01%-5%;
Zinc PCA: 0.01%-4.0%;
Magnesium chloride: 0.01%-2%;
Potassium chloride: 0.01%-2%;
Calcium chloride: 0.01%-2%;
Sodium chloride: 0.01%-2%; and
remaining water and excipients.

What is claimed is:

1. A topical composition, comprising:
a. at least four vitamins;
b. at least one electrolyte;
c. at least one mineral;
d. and at least one excipient.

2. The topical composition of claim 1, comprising at least five, at least six, at least seven or more vitamins.

3. The topical composition of claim 1, wherein the vitamin is plant derived.

4. The topical composition of claim 1, wherein the vitamins are selected from a group consisting of linoleic acid and linolenic acid fatty acids (vitamin F), niacinamide (vitamin B3), sodium ascorbyl phosphate (vitamin C), cholecalciferol (vitamin D3), phytomenadione (vitamin K₁), Calcium D-pantothenic acid (vitamin B₅), tocopheryl acetate (vitamin E acetate), tocopherol (vitamin E), or a suitable derivative or variant thereof.

5. The topical composition of claim 4, wherein the vitamins are present at the following range of weight percentage (wt/wt %) of the topical composition:
linoleic acid and linolenic acid fatty acids (vitamin F): 0.01-2%;
niacinamide (vitamin B3): 0.01%-5%;
sodium ascorbyl phosphate (vitamin C): 0.01%-20%;
cholecalciferol (vitamin D3): 0.01%-2%;
phytomenadione (vitamin K₁): 0.01%-2%;
D-Calcium pantothenic acid (vitamin B₅): 0.01%-5%;
tocopheryl acetate (vitamin E acetate): 0.01%-5%; and
tocopherol (vitamin E): 0.01%-5%.

6. The topical composition of claim 1, wherein the at least one electrolyte comprises a magnesium salt, a potassium salt, and a calcium salt, or any combination thereof.

7. The topical composition of claim 6, wherein the at least one electrolyte comprises magnesium chloride, potassium chloride, calcium chloride, or any combination thereof, and the at least one electrolyte is present in an amount of about 0.01 to about 2 wt/wt %.

8. The topical composition of claim 1, wherein the at least one mineral is zinc pyrrolidone carboxylic acid (PCA).

9. The topical composition of claim 8, wherein the zinc PCA is present in an amount of about 0.01 to about 4 wt/wt %.

10. The topical composition of claim 1, wherein the excipients comprise any one or more of humectants, emollients, solvents, oils, fats, thickening agents, buffers, preservatives, coloring agents, fragrance, or any combination thereof.

11. The topical composition of claim 10, wherein the excipients comprise water, glycerin, glyceryl stearate, Aloe barbadensis leaf juice, sorbitan oliviate, hexylene glycol, jojoba esters, magnesium stearate, corn oil, capryly glycol, allantoin, Phenoxyethanol, caprylyl glycol, ethylhexylglycerin, or any combination thereof.

12. The topical composition of claim 11, wherein the excipients are present in the range of about 80 wt % to about 90 wt % of water, about 1.5 wt % to about 2 wt % of glycerine, about 1.5 wt % to about 2.5 wt % of glyceryl stearate, about 1 wt % to about 3 wt % of Aloe barbadensis leaf juice, about 0.5 wt % to about 1.5 wt % of sorbitan oliviate, less that about 1 wt % of hexylene glycol, less that about 1 wt % jojoba esters, less that about 1 wt % magnesium stearate, less that about 1 wt % of corn oil, less that about 1 wt % allantoin, less that about 1 wt % capryly glycol, less that about 1 wt % phenoxyethanol, and less that about 1 wt % ethylhexylglycerin.

13. A topical composition comprising by wt/wt %:
Water (Aqua): 82-90%;
Glycerin: 3-6%;
Glyceryl Stearate: 1.5-2.5%;
Aloe barbadensis Leaf Juice: 1-3%;
Sorbitan Olivate: 0.5-1.5%;
Hexylene Glycol: <1%;
Linoleic Acid: 0.01-2%;
Linolenic Acid: 0.01-2%;
Niacinamide: 0.01%-5%;
Sodium Ascorbyl Phosphate: <1%;
Allantoin: <1%;
Sodium Hyaluronate: <1%;
Cholecalciferol: 0.01%-2%;
Jojoba Esters: <1%;
Magnesium Stearate: <1%;
Phytonadione: 0.01%-2%;
Zea mays (Corn) Oil: <1%;
D-Calcium Pantothenate: 0.01%-5%;
Zinc PCA: <1%;
Carbomer: <1%;
Magnesium Chloride: <1%;
Potassium Chloride: <1%;
Sodium Chloride: <1%;
Calcium Chloride: <1%;
Phenoxyethanol: <1%;
Tocopheryl Acetate: 0.01%-5%;
Caprylyl Glycol: <1%;
Ethylhexylglycerin: <1%; and
Tocopherol: <1%.

14. The topical composition of claim 1, wherein the topical composition is administered to the surface on the subject to prevent, reduce, or treat one or more of fine lines, wrinkles, moisture loss, hyperpigmentation, uneven skin tone, crepe skin, collagen loss, oxidation, uneven skin tone, skin redness, or free radical formation.

15. The topical composition of claim 1, wherein the topical composition is administered to the surface on the subject to enhance one or more of hydration, passage for absorption, skin brightness, or healthy glow.

16. The topical composition of claim 1, wherein the composition is in the form of a solids, liquids, suspensions, semisolids (such as creams, gels, pastes or “sticks”), powders or finely dispersed liquids such as sprays or mists.

17. A method of improving the condition of the skin of a subject in need thereof comprising, applying to the subject the topical composition of claim 1.

18. The method of claim 17, wherein the subject is a human.

19. A kit for skincare comprising the topical composition of claim 1, and instructions for use.
